(19) United States
(12) Patent Application Publication
(10) Pub. No.: US 2024/0148459 A1
(43) Pub. Date: May 9, 2024

(54) A MOBILE SELF-POWERED MEDICAL SERVICES TROLLEY FOR USE IN AN OPERATING THEATRE

(71) Applicant: SC MEDICAL PTY LTD, Queensland (AU)

(72) Inventors: Craig Walton, Queensland (AU); Nicholas Ullrich, Queensland (AU)

(21) Appl. No.: 18/282,367

(22) PCT Filed: Mar. 17, 2022
(86) PCT No.: PCT/AU2022/050231

§ 371 Date: Sep. 15, 2023

(30) Foreign Application Priority Data
Mar. 17, 2021 (AU) 2021900767

(51) Int. Cl. A61B 50/13; A61B 17/00; A61B 90/00; G16H 40/40

(52) U.S. Cl. CPC A61B 50/13; A61B 17/00; A61B 90/361; G16H 40/40; A61B 2017/00221; A61B 2017/00548; A61B 2017/00734; A61B 2090/373; A61B 2217/005

(57) ABSTRACT

There is provided a mobile self-powered medical services trolley for use in a surgical operating theatre. The trolley includes a portable power supply in the form of battery system that powers the trolley without the need for an external power source (such as mains power) to be wired to the trolley. The trolley includes a plurality of electrically powered medical instruments and other electrically powered devices integrated onto the trolley and powered by the battery system for use in medical procedures carried out in the theatre. The plurality of medical instruments includes: a suction instrument; a gas supply apparatus; and wireless data communication unit in communication with a central computer network, for both sending and receiving data. The trolley further includes a multimedia input in the form of a connector port which is configured to connect to a video capture device.

### TECHNICAL FIELD

The present disclosure relates to a mobile self-powered medical services trolley for use in an operating theatre. The present disclosure has applications in the field of medical devices and equipment specifically for use in surgical procedures including integrating with other existing equipment in an operating theatre but also for use in other medical activities.

While some embodiments will be described herein with particular reference to that application, it will be appreciated that the invention is not limited to such a field of use, and is applicable in broader contexts.

### BACKGROUND

Any discussion of the background art throughout the specification should in no way be considered as an admission that such art is widely known or forms part of common general knowledge in the field.

Healthcare facilities are large and complex with many critical building services. In particular, operating theatres within those facilities are critical care areas in hospitals in which workflow and access to equipment is essential to the ability of clinical staff performing operations to minimise risk to patient safety and maximise efficiency of the operating theatre.

As new technologies are developed, there has been a significant increase in the amount of equipment in an operating theatre. This has resulted in a significant increase in the size of an operating theatre. An example of this is that in Australia the Health Facility Guidelines now specify a minimum room size of 60 sqm up from just 40 sqm as was the previous standard. Further, the cost of installation and maintenance of the building has increased in line with this.

Generally speaking, operating theatres are known to include or be supplied with many medical devices or instruments including high purity pressure adjustable air conditioning, colour accurate high intensity luminaries, various sources of high reliability electrical feeds, high purity medical gases and immense structured audio video cabling located in the ceiling space. Poor equipment access causes undue stress on clinical staff.

Also significant to the layout and equipment in operating theatres is the increase to minimally invasive surgery and the use of cameras and visualisation through keyhole incisions. This has increased the need for more monitors within the operating theatre for critical staff to view an operation. Equipment for minimally invasive surgery was originally on a moveable trolley, known as an “equipment stack”, and typically includes a camera, light source and insufflator with a monitor on top, an example of which is shown in FIG. 1. A diathermy apparatus was typically mounted on its own trolley. The issues with this known system include:
The trolley having to be positioned such that a surgeon could view the monitor, which typically resulted in the equipment stack having to be positioned on the opposite side of the patient to the surgeon. Therefore, all the patient leads had to be routed under the table to the surgeon to avoid the sterile operating site.
Electrical cables were run across the floor to services on the wall to power the equipment creating an occupational health and safety issue of a trip hazard in a very busy environment.
Other trolleys were required to provide suction and for other equipment such as diathermy and patient warmers all of which have associated power cables and/or other infrastructure connections.

To overcome these issues, medical equipment manufacturers began mounting equipment on equipment pendants or booms (as shown in FIG. 2) and hanging multiple monitors within the operating theatre to provide better viewing for the surgeon, the surgeon's assistant nurses and other clinical staff. This evolved into what is known as an integrated theatre (shown in FIGS. 3A and 3B) which has the ability to route video signals from multiple sources to monitors internally within the theatre and also external to the theatre, for example, for training purposes.

Referring to FIG. 3A which illustrates an example of integrated theatre or suite, the integrated theatre has allowed equipment to be placed on shelves so as to minimise the trip hazards created by the need for cables to run from trolleys across the theatre to power and other services. Video signals are routed through the arm of the pendant to a video routing hub that allows selection from different inputs to display on the monitors that hang from independent arms. The monitors typically hang either side of an operating table.

The issues with this system are clearly viewable in FIG. 3B, which depicts an example of a 49.95 sqm operating theatre layout and the limitations of movement for an equipment pendant. As shown, the area of movement of the equipment pendant (a shaded circle in FIG. 3B) is limited to approximately a circle of about 10 sqm (as the three joints of the pendant are only able to rotate 330 degrees) and, therefore, covers the right side of the patient but would have difficulties accessing the foot end, heads end and the left side of the patient. As such many theatres that have integrated suites still employ a number of equipment trolleys which takes up more floor space in the operating theatre and reintroduces cables and/or hoses, thereby essentially negating any advantages of using the pendant system.

Further, with the advent of higher video quality, upgraded cabling is required to allow transmission of high bandwidth video formats, which is very difficult and costly. Often hospitals will upgrade monitors believing they will get a better picture. However, if the cabling and video router are not equipped to handle the new format, the image quality is no better.

The design of workflow within an operating theatre is essential to good patient outcomes.

### SUMMARY

It is an object of the present invention to overcome or ameliorate at least one of the disadvantages of the prior art, or to provide a useful alternative.

In accordance with a first aspect of the present invention there is provided a mobile self-powered medical services trolley for use in an operating theatre, the trolley including:
a portable power supply;
a plurality of instruments integrated onto the trolley and powered by the power supply for use in medical procedures, the plurality of instruments including:
   a suction instrument;
   a gas supply apparatus; and
   wireless data communication unit in communication with a central computer network; and
a multimedia input.

In an embodiment, the power supply includes at least one battery.

In an embodiment, the power supply includes a plurality of batteries connected in parallel.

In an embodiment, the at least one battery is rechargeable.

In an embodiment, the suction instrument includes a vacuum pump.

In an embodiment, the gas supply apparatus includes a carbon dioxide supply apparatus. In an embodiment, the carbon dioxide supply apparatus includes a carbon dioxide storage cylinder.

In an embodiment, the gas supply apparatus includes at least two gas storage cylinders containing the same medical gas such that the gas supply apparatus is initially connected to one of the at least two gas storage cylinders, the gas supply apparatus configured to automatically change connection to another of the at least two gas storage cylinders when the initially connected gas storage cylinder is depleted.

In an embodiment, the gas supply apparatus includes a plurality of gas storage cylinders, each cylinder containing a different medical gas.

In an embodiment, the multimedia input includes a connector port for connecting to an external video capture device. In an embodiment, the connector port is one of: an HDMI port; and an SDI port.

In an embodiment, the multimedia input includes an integrated video capture device. In an embodiment, the integrated video capture device includes at least one video camera. In an embodiment, the at least one video camera is a high-definition video camera.

In an embodiment, the wireless data communication unit utilises a wireless ethernet connection.

In an embodiment, the wireless data communication unit includes a transmitter for communicating with a remote receiver connected to the central computer network, the central computer network including a standalone video routing system such that data from the video capture device is inputted into the standalone video routing system for display within the surgical theatre.

In an embodiment, the trolley further includes a location positioning system for allowing location detection of the trolley. In an embodiment, the location positioning system includes an integrated radio receiver.

In an embodiment, the trolley further includes a line isolation safety monitor for monitoring leakage to ground current.

In accordance with a second aspect of the present invention there is provided a mobile self-powered medical services trolley for use in a surgical theatre, the trolley including:
a portable power supply; and
a plurality of devices integrated onto the trolley and powered by the power supply for use in medical procedures, the plurality of instruments to supply the following functions:
   suction;
   gas supply;
   video capture input; and
   wireless data communication.

In accordance with a third aspect of the present invention there is provided a method for providing self-powered suction, gas supply and video capture to a surgical theatre including providing a medical services trolley according to the first or second aspects.

In accordance with a fourth aspect of the present invention there is provided a central computer network for displaying a visual input on at least one monitor within a surgical theatre, the network including a standalone video routing system for receiving video data from a medical services trolley according to the first or second aspects.

Other aspects of the present disclosure are also provided.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

### BRIEF DESCRIPTION OF THE DRAWINGS

One or more embodiments of the present disclosure will now be described by way of specific example(s) with reference to the accompanying drawings, in which:

FIG. 1 is an example of a known movable equipment stack of the prior art;

FIG. 2 is an example of a known equipment pendant of the prior art;

FIG. 3A is an example of a known integrated operating theatre of the prior art including a known equipment pendant of the prior art;

FIG. 3B is a plan view of the known integrated operating theatre of FIG. 3A;

FIG. 4 is a conceptual block representation of a mobile self-powered medical services trolley in a surgical operating theatre according to an embodiment of the invention;

FIG. 5A is a front view representation of the trolley of FIG. 4;

FIG. 5B is a side view representation of the trolley of FIG. 4; and

FIG. 6 is a schematic representation of the trolley of FIG. 4.

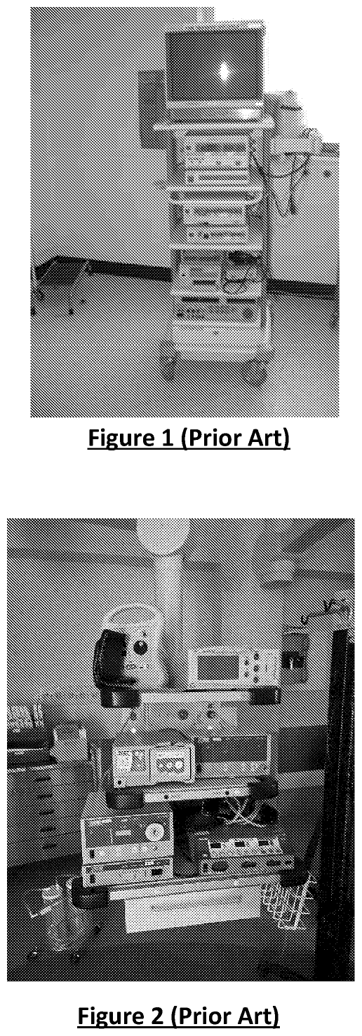

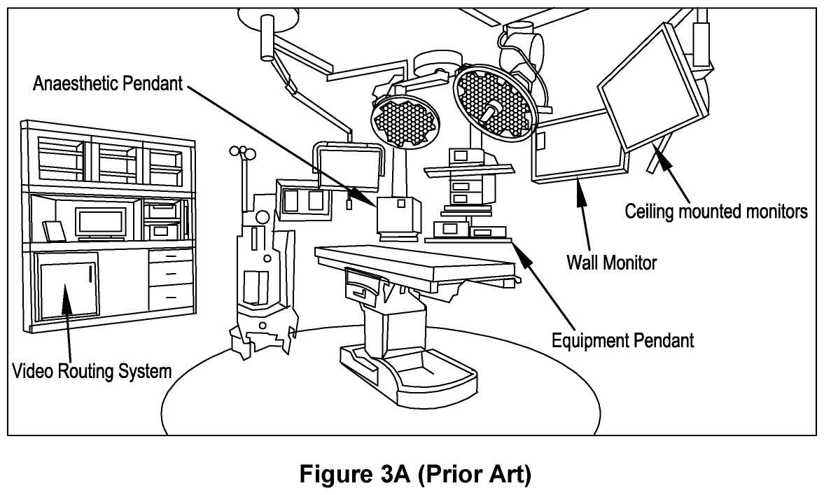

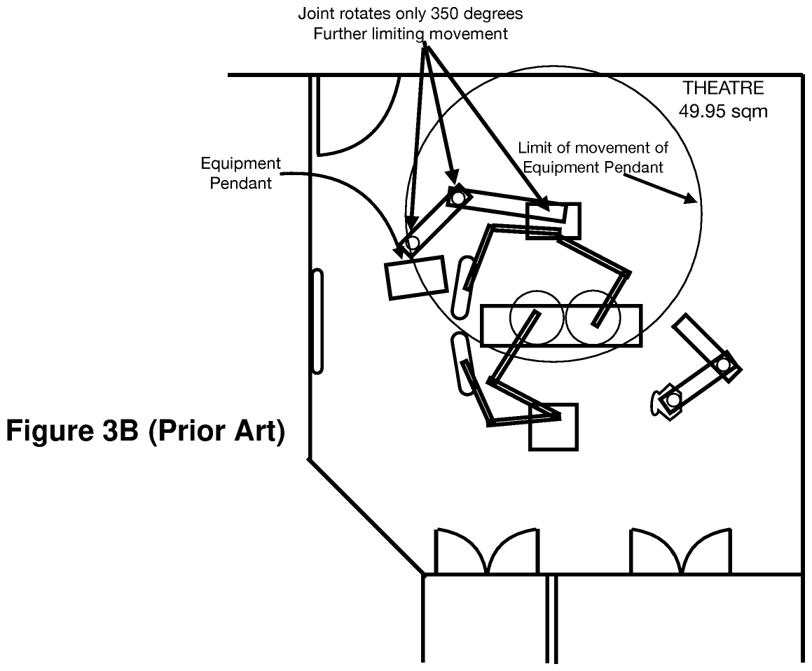

### DETAILED DESCRIPTION

Where applicable, steps or features in the accompanying drawings that have the same reference numerals are to be considered to have the same function(s) or operation(s), unless the contrary intention is expressed or implied.

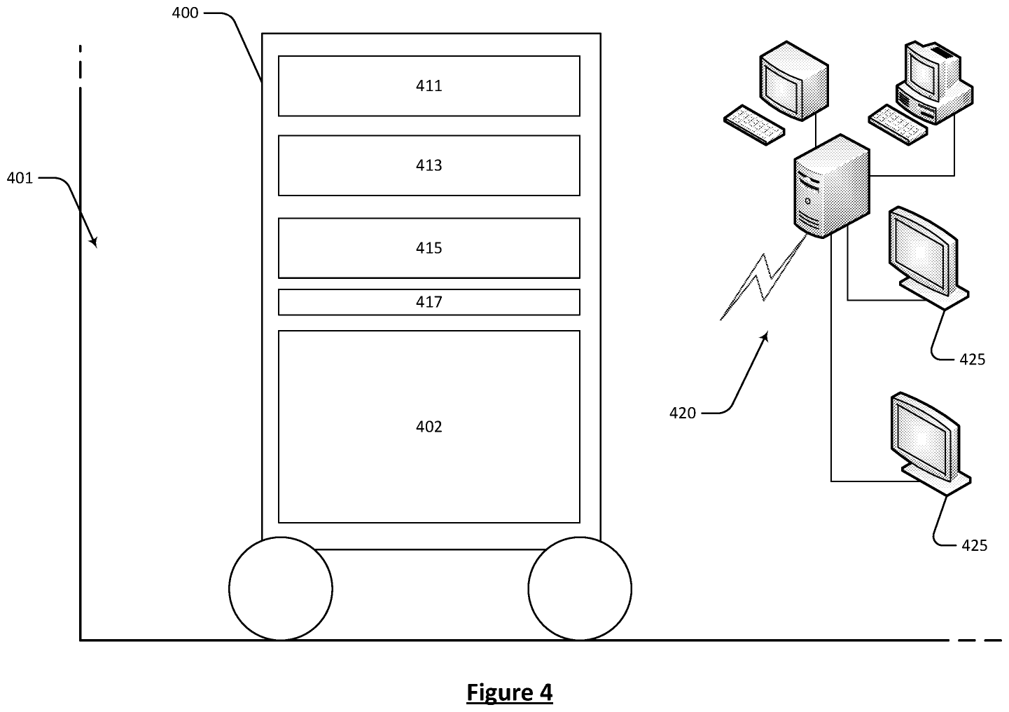

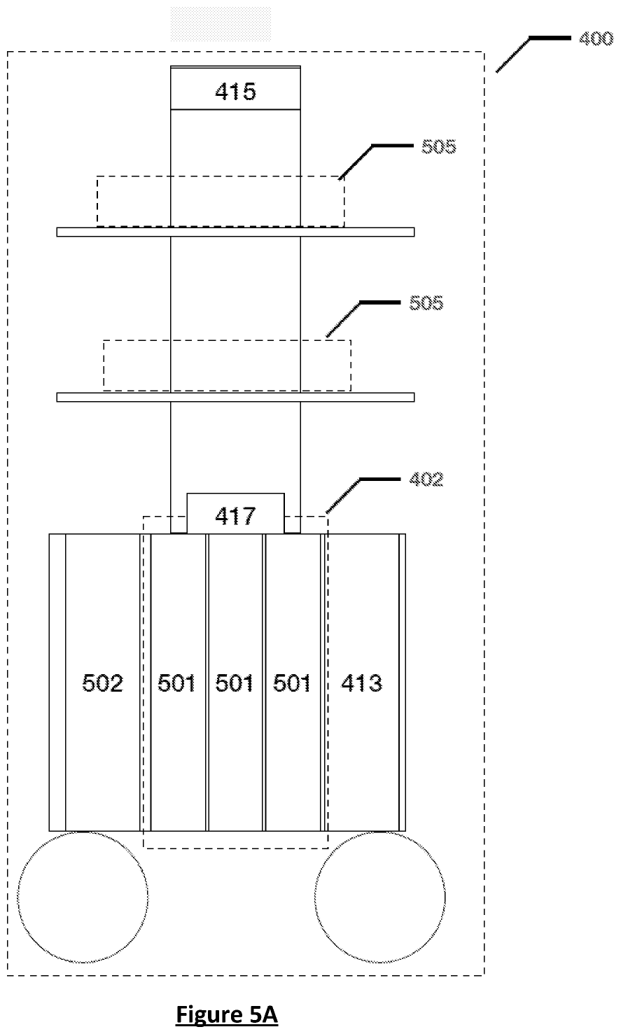

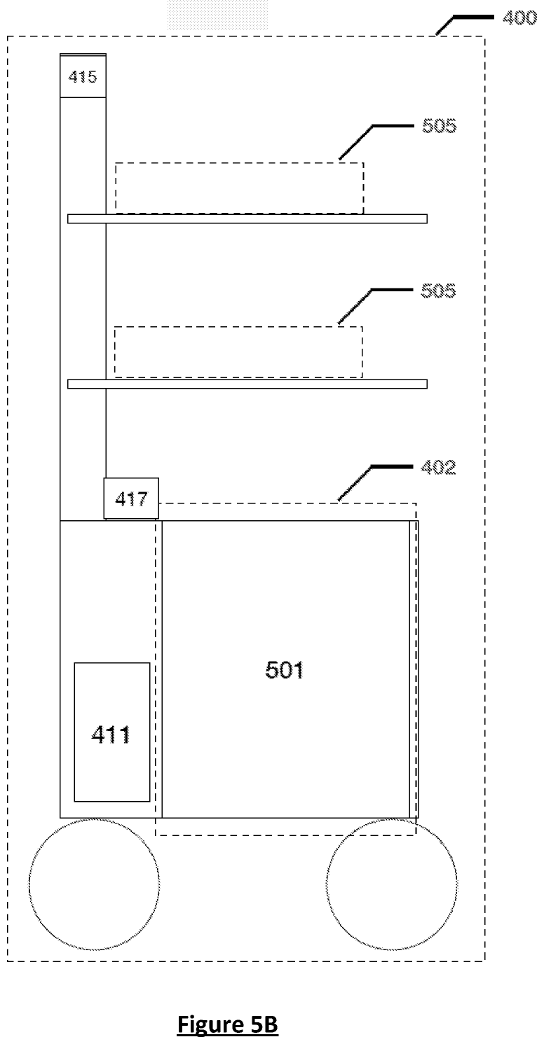

Referring to initially to FIGS. 4, 5A and 5B there is provided a mobile self-powered medical services trolley 400 for use in a surgical operating theatre 401. Trolley 400 includes a portable power supply in the form of battery system 402 that powers trolley 400 without the need for an external power source (such as mains power) to be wired to trolley 400. Trolley 400 includes a plurality of electrically powered medical instruments and other electrically powered devices integrated onto trolley 400 and powered by battery system 402 for use in medical procedures carried out in theatre 401. The plurality of medical instruments includes: a suction instrument 411 for supporting medical equipment; a gas supply apparatus 413 for driving medical equipment; and wireless data communication unit 415 in communication with a central computer network 420, for both sending and receiving data. Trolley 400 further includes a multimedia input in the form of a connector port 417 which is configured to connect to a video capture device.

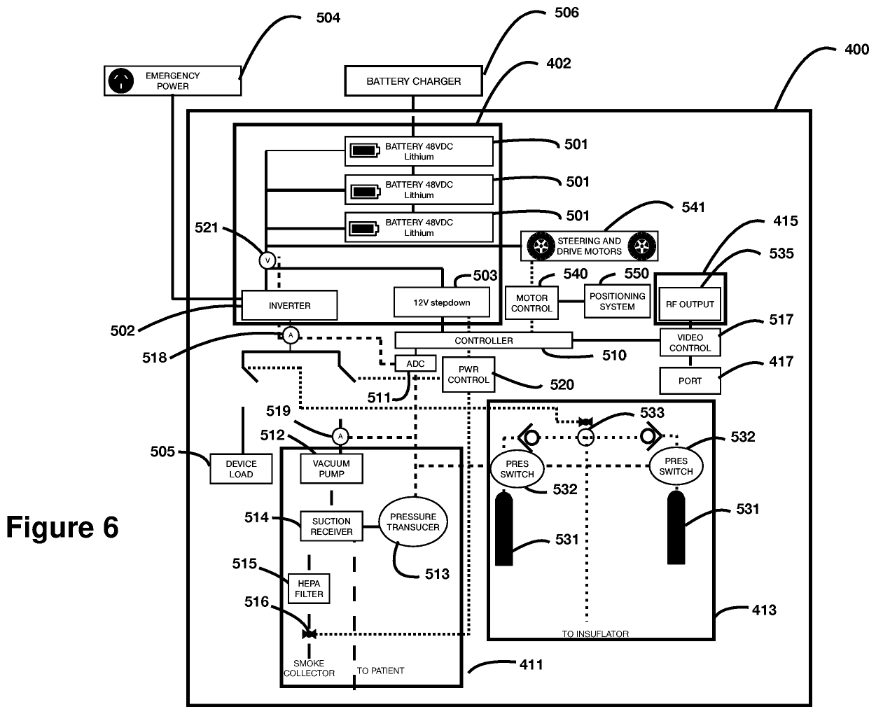

Referring to FIGS. 5A and 6, battery system 402 includes three rechargeable 3.3 Kilowatt 48 Volt DC lithium ion batteries 501 that are connected in parallel. Battery system 402 is able to provide in excess of 10 hours of run time based on typical equipment stacks (such a stack being represented by device load 505 and typically including a scope camera device and light source, and an insufflator at minimum, but may also include a printer, a video capture device and an irrigation pump). Further, although the system could feasibly function on a single battery for a period of time, the preferred embodiments having multiple batteries ensure at least 10 hours run time of trolley 400 without requiring recharge and provide redundancy due to the presence of multiple batteries 501. As will be appreciated by those skilled in the art, specific runtimes will vary depending on the final equipment setup. The desired runtime is such that trolley 400 can be recharged at the end of each day.

In other embodiments more than three of batteries 501 are used. In other yet embodiments, different voltage batteries are used. In yet other embodiments, non-rechargeable batteries, such as lithium batteries and alkaline batteries. Battery system 402 also includes an AC inverter 502 and a 12 Volt stepdown transformer 503 both independently electrically coupled to batteries 501 for providing AC power and low voltage power source to different components of trolley 400 as required.

Inverter 502 converts the direct current (DC) voltage supplied by batteries 501 to a low voltage alternating current (AC). Inverter 502 has a provision to be connected to an emergency power source 504 in the form of an external low voltage AC source should batteries 501 completely deplete while in use. The external low voltage AC source is fed into a one-to-one isolation transformer (not shown) of inverter 502 to void any earth leakage currents. When a connection is made to the external low voltage AC source, battery system 402 and step-up function essential disabled by disconnection of batteries 501.

Batteries 501 are rechargeable via a temporary connection to a battery charger charging dock 506. Charging dock 506 converts low voltage AC to extra low DC to charge batteries 501 at their nominated charging voltage. Charging dock 506 is mounted in a fixed position on a wall within operating theatre 401 or in a fixed position on a wall of a central storage room. Further, in some embodiments, the central storage room contains a bank of docking points for engaging with multiple trolleys simultaneously.

Referring to FIGS. 5B and 6, trolley 400 includes a controller 510 electrically coupled to the low voltage power source of battery system 402. Controller 510 includes a central trolley processor for controlling the functionality of the components of trolley 400. Each of suction instrument 411, gas supply apparatus 413 and wireless data communication unit 415 are controlled by controller 510. Controller 510 includes a plurality of external controls that actuate each of the functions of trolley 400. The external controls include a touchscreen interface. In other embodiments, the external controls include plurality of buttons and dials. In yet other embodiment, the external controls are a combination of mechanical buttons and dials, touch-sensitive buttons and a touchscreen. In yet other embodiments, controller 510 is configured to receive input commands from a remote controller via wireless data communication unit 415. Controller 510 is coupled to a power controller 520 for controlling power from battery system 402 being supplied to the plurality of electrically powered medical instruments and other electrically powered integrated devices.

Controller 510 is coupled to suction instrument 411 via an analogue to digital converter (ADC) 511 to control the activation and deactivation of suction as well as the level of suction pressure. Suction instrument 411 includes an internal vacuum pump 512 and a pressure transducer 513 for monitoring the level of suction that feeds into a suction receiver 514. Suction receiver 514 includes a suction nozzle or suction outlet (not shown) for suctioning matter from a patient. The level of suction pressure is adjusted via a solenoid valve (not shown). Suction instrument 411 includes a suction waste nozzle or outlet (not shown, as will be appreciated by those skilled in the art this component will vary depending on the country standard outlet) that is connectable to existing hospital waste containers.

In alternate embodiments, suction instrument 411 includes a connection to building suction in case of emergency.

Furthermore, suction receiver 514 is also connected to a high-efficiency particulate air (HEPA) 515 filter that is subsequently connected to a suction intake 516 for evacuating smoke and other airborne particulate matter within operating theatre 401. This is particularly pertinent if utilising diathermy or a medical laser for removal of any plumes generated that maybe dangerous to those in operating theatre 401. In preferably embodiments, suction receiver 514 activates suction intake 516 when processes such as diathermy or laser usage is utilised. In embodiments, controller 510 will automatically detect the use of diathermy or medical laser and automatically activate suction intake 516. In such embodiments, controller 510 is coupled to sensors to detect such usage. In other such embodiments, trolley 400 includes a diathermy apparatus or medical laser which, once activated, with cause controller 510 to activate suction intake 516.

There is also included a first ammeter 518 coupled directly downstream of inverter 502 and a second ammeter 519 coupled directly upstream of suction instrument 411 for measuring load on battery system 402. Further, there is included a voltmeter 521 coupled directly downstream of batteries 501 for measuring battery level.

Referring to FIG. 6, controller 510 is also coupled to gas supply apparatus 413. Gas supply apparatus 413 includes two carbon dioxide gas vessels in the form of carbon dioxide gas storage cylinders 531 each having a pressure switch 532 which is connected to an automatic changeover system 533 that is subsequently connected to an insufflator (not shown). In preferred embodiments, gas storage cylinders 531 are size C or D. In other embodiments, gas storage cylinders 531 are size G. In yet other embodiments, gas storage cylinders 531 are a customised size specifically built for use with trolley 400. Controller 510 will generally control which of gas storage cylinders 531 is presently in use through automatic changeover system 533. In the illustrated embodiment, gas storage cylinders 531 both contain carbon dioxide gas such that gas is drawn from one of gas storage cylinders 531 for use as a first supply and when the first supply is depleted, controller 510 causes automatic changeover system 533 to draw gas for use from the other of gas storage cylinders 531 which is used to as a back-up supply to the first supply. Automatic changeover system 533 is configured to automatically change gas supplies from the first supply to the back-up supply once the first supply is depleted. Controller 510 trolley monitors the supply of each of gas storage cylinders 531 and provides an alert when the first supply has been depleted and only the back-up supply is available.

The one of gas storage cylinders 531 used as the first supply is releasably mounted to trolley 400 such that it can be removed and replaced with an identical gas storage cylinder without the need for interrupting the supply of carbon dioxide gas or the sterile field. In some embodiments, trolley 400 includes an external gas inlet preferably positioned on the rear of trolley 400 for emergency connection to an external source of carbon dioxide gas to be used in the event the gas supply within trolley 400 is disrupted due to a system failure.

In other embodiments, only one gas storage cylinder 531 is used. In other embodiments, more than two gas storage cylinders 531 are used.

In other embodiments gas supply apparatus 413 includes multiple gas storage cylinders containing different medical gasses. For example, one of the gas storage cylinders contains carbon dioxide and another of the gas storage cylinders contains another medical gas such as nitrous oxide or oxygen.

Referring to FIGS. 4, 5A, 5B and 6, controller 510 is also coupled to wireless data communication unit 415 which includes a radio frequency (RF) output 535. Wireless data communication unit 415 utilises a wireless ethernet connection for wirelessly communicating with central computer network 420. More specifically, wireless data communication unit 415 includes a transmitter for communicating with a remote receiver connected to central computer network 420. In other embodiments, the remote receiver is integrated with and therefore part of central computer network 420.

Controller 510 is also coupled to a motor controller 540 which is subsequently coupled to steering and drive motors collectively denoted as reference 541. Motor controller 540 controls the functioning of steering and drive motors 541 for controlling the movement of trolley 400 driven by drive motors and directed by steering motors. Steering and drive motors 541 are powered by battery system 402 via inverter 502. Steering and drive motors 541 are connected to motorised wheels such that weight issues are overcome as this alleviates the need to manually push the heavy trolley particularly over relatively long distances.

Trolley 400 further includes a location positioning system 550 coupled to motor controller 540 for allowing location detection of trolley 400 within a facility containing multiple operating theatres and one or more storage rooms where trolleys may be located. Location positioning system 550 includes an integrated radio receiver (not shown) for communicating the location of trolley 400 with central computer network 420. In some embodiments, controller 510 is configured to deduce location information of trolley 400 within the facility by way of signal strength detection techniques. In other embodiments, other technologies are utilised to deduce location information of trolley 400, such as signal direction and/or signal timing detection techniques. In yet other embodiments, a combination of the aforementioned technologies are utilised to deduce location information. The location information is used by trolley 400 for configuration purposes such as a specific doctor's positioning and orientation preferences of trolley 400 for a specific type of operation within operating theatre 401, video routing preferences of clinical staff, and network selection.

In other embodiments, the location positioning system is integrated with wireless data communication unit 415.

In further embodiments, trolley 400 utilises a Real Time Location System (RTLS) as part of location positioning system 550 to locate trolley within the facility and to connect trolley 400 to a hospital Patient Information System. RTLS allows automatic configuration of trolley 400 by controller 510 and allows verification of trolley 400 and, if applicable, other trolleys within operating theatre 401 that the trolleys are appropriate for the upcoming scheduled type of medical procedure linking to surgeon's equipment preferences for that specific procedure.

In some embodiments, trolley 400 has computer vision capabilities (utilising motor controller 540, the video capture device connected to connector port 417 and in certain embodiments controller 510) to enable trolley 400 to autonomously move to follow a user, such as a doctor or drive itself to charging dock 506. The computer vision capabilities include video tracking and object recognition, amongst others.

In some embodiments, trolley 400 is fitted with LEDs that are illuminated with a specific colour to indicate the type of equipment on the trolley 400, which can therefore be recognized from a distance.

In some embodiments, trolley 400 will utilise RTLS to disable the LEDs when entering a designated area such as a theatre. More specifically, RTLS is able to recognise when trolley 400 enters the designated area and provide a signal to controller 510 to disable the LEDs upon such an event occurring.

Controller 510 includes a video control unit 517 that is connected to connector port 417. In preferred embodiments, connector port 417 is an HDMI port or an SDI port. In other embodiments, trolley 400 includes a plurality of connector ports each of which are different port connector interfaces. Connector port 417 is configured to receive an HDMI or SDI cable from a video source such as an external video capture device including a camera (not shown) whereby the images captured by the video capture device are inputted for communication by wireless data communication unit 415 to central computer network 420. This communication utilises 60 Ghz wireless transmission for providing near zero latency, real 4 k transmission, and robust protection from radio frequency interference (which is typically high in operating theatre 401 due to radio frequency generators such as diathermies). The 60 Ghz wireless transmission is utilised as it is limited to transmission within operating theatre 401 as it does not penetrate the walls of operating theatre 401, thereby not causing interference between adjacent operating theatres. In preferred embodiments, the video capabilities integrate into any existing central video routing system of central computer network 420. In other embodiments, different wireless wavelengths and transmissions rates other than 60 Ghz are utilised. In embodiments, trolley 400 includes an emergency video connection via a compatible video sample with the central video system is supplied in case of a transmission failure. In yet other embodiments, in addition to connector port 417, wireless data communication unit 415 receives wireless transmission and/or routing of video signals from the video capture device powered by trolley 400.

The video capture device is connected to a scope camera device such as an endoscopic camera system which, as will be appreciated by a person skilled in the art, includes a camera head connected to a scope (the scope camera device controls the camera head functions like white balance and aperture and also converts the camera video into a video signal). The video capture device plugs into the video output of the scope camera device and records the video (like PVR) or saves video files to a USB thumb drive.

The video source (along with the scope camera device, camera head, and scope, if applicable) will form part of device load 505 which is powered by battery system 402. In other embodiments, the video source is powered externally and the video feed from the video source is inputted into to video control unit 517 for routing to the existing central video routing system of central computer network 420.

In some other embodiments, the video capabilities of trolley 400 is a standalone closed system.

In other embodiments trolley 400 includes an integrated video capture device in the form of a high-definition video camera. In yet other embodiments, the integrated video capture device is other than a high-definition video camera. In yet other embodiments, the video camera is standard definition. In yet other embodiments, the video camera is ultra-high definition.

In embodiments, the central video system of central computer network 420 receives data from the video capture device for display on one or more monitors 425 (in the embodiment of FIG. 4, two monitors are utilised) within operating theatre 401. In other embodiments, trolley 400 includes an integrally mounted display monitor for direct wired connection to the external video capture device.

In addition to the video source, device load 505 includes all external equipment for use in an operation that is required to be in close proximity to a patient and clinicians and that requires power from battery system 402. In various embodiments, device load 505 further includes, amongst others:
A light source associated with the video source;
An insufflator irrigation unit;
A printer;
A diathermy apparatus;
An external smoke evacuator;
An irrigation pump (for pumping sterile fluids into a patient for flushing, for example, debris clear from joints);
A patient warmer; and
An infusion device.

In alternate embodiments, one or more of the above device load components is integrated into trolley 400.

Trolley 400 further includes a line isolation safety monitor (not shown) for monitoring leakage to ground current such that the detection of a leakage to ground current will cause the trolley to electrically isolate from battery system 402 to prevent electrocution of a person (either medical professional, patient or another person).

Referring to FIG. 6, there are a number of non-solid connector lines that are included to illustrate the communication of different types of functionality of trolley 400. Those are:
Input/feedback measurements (for power, suction, pressure, amongst others) denoted by the line of long dashes each separated by two dots.
Control signals denoted by the line of long dashes.
Gas supply system channels denoted by the line of short dashes.
Suction system channels denoted by the dotted line.

In other embodiments, trolley 400 is used in rooms other than surgical operating theatre, including endoscopy rooms, procedure rooms, resuscitation rooms and interventional imaging laboratories (such catheterization laboratories, angiography laboratories). In embodiments, trolleys for use in different rooms include certain specific features that are applicable to the activities in those rooms.

As noted above, trolley 400 is used in operating theatre 401. Prior to commencing an operating procedure, trolley 400 is moved into operating theatre 401 and positioned in a desired location within operating theatre 401. Trolley 400 can either be manually pushed or pulled when its wheels are not locked or it can be moved by controlling steering and drive motors 541 or a combination of the two (for example, moving larger distances is done using steering and drive motors 541 and the final small adjustments of position done manually). As mentioned above, the position and orientation of trolley 400 will be based on the preference of the doctor (or doctors) and how they like operating theatre 401 set up, along with the particular procedure and the general layout of operating theatre 401 including where display monitors 425 are located, amongst others. For example, a doctor may wish to stand on a certain side of the patient due to either personal preference or ease of accessibility of the area of interest of the patient and, therefore, nurses and other medical professionals within operating theatre 401 will have to work around this and the position and orientation of trolley 400 will be affected by the position of the people in operating theatre 401. Further, in embodiments where trolley 400 includes an integrally mounted display monitor, trolley 400 will need to be in a position where the display monitor can be seen by the doctor and other relevant personnel that need to view the monitor.

Once the requisite preparation is completed, including positioning trolley 400 in place, the operation will commence. Given the mobility of trolley 400, it can be easily repositioned during the operation as required.

Finally, once the operation (or a series of shorter operations) are completed, trolley 400 is moved to and coupled to charging dock 506 for batteries 501 to be recharged so that they are fully charged (or at least mostly charged) prior to the next use of trolley 400. In alternate embodiments, batteries 501 can be exchanged while trolley 400 is in use, thereby effectively increasing supply capacity to extend run time.

In further embodiments, charging dock 506 includes a high-pressure line and is also configured to automatically refill gas storage cylinders 531 via the high pressure line.

### Advantages of Detailed Embodiments

It will be appreciated that the embodiments of trolley 400 described herein are advantageous over known devices and systems as they are able to power mains powered medical electrical equipment allowing integration with other equipment in the operating theatre and/or hospital without being connected to the hospital electrical supply or other services such as gas supplies, network and video routing systems. Embodiments of trolley 400 have been devised to increase flexibility within the operating room by building a trolley that contains a rechargeable battery system and an inverter to supply power to medical equipment, thereby not requiring wiring to the operating theatre. The trolley provides efficiencies of being mobile while eliminating trip hazards caused by devices requiring cables and tubes to building supplies. The trolley includes the following advantageous features in order to operate independently”:
Power via a battery supply and inverter.
Suction via a vacuum pump powered from the battery supply.
Carbon Dioxide via storage cylinders and, in embodiments, an automated change over to back-up cylinder.
Data and video transmission wireless via a transmitter to a receiver to input to a central video routing system or other systems of the hospital.
Wireless ethernet connection for connecting medical equipment to the hospital network or other systems of the hospital.
Line isolation monitor on the transformer in the inverter to monitor the leakage to ground (earth leakage) to ensure the integrity of isolation and maintain patient and medical professional safety.
Location positioning system to identify trolley location within the facility for, amongst others, configuration of equipment, routing of video signals and allowing seamless transition from one theatre to another.
In some embodiments, motorised drive system and computer vision to autonomously follow a user.

These features allow the trolley to be positioned anywhere in an operating theatre eliminating all the issues with the traditional methods discussed in the background section. The features of trolley 400 allow other medical equipment to be either positioned on or adjacent to trolley 400 to utilize those features of trolley 400 to operate as per the equipment manufacturer's specifications without a direct connection to the hospital infrastructure.

The isolation transformer is essential to patient safety especially in the situation where the battery supply fails and the trolley is required to be plugged into the hospital main supply. Further, the steering and drive motors will alleviate occupational health and safety issues that arise due to the heavy weight of the trolley in that the motorisation will absolve the need to manually move the heavy trolley by pushing and pulling. This takes away the risk of injury to a person that would otherwise have to manually move the heavy trolley.

Another advantage of the trolley is that is can be utilised in new facilities as well as older theatres where easily adding medical services into an existing older building is impractical. Regarding newer theatre, these can be built smaller and with less services, thereby reducing building costs. The increased flexibility of movement will directly relate to increases in efficiencies in hospital and improved patient outcomes.

The trolley provides ‘off grid’ essential services in the operating theatre to allow flexibility in positioning of equipment while maintaining patient safety and security of supply.

### Conclusions and Interpretation

Throughout this specification, where used, the term “element” is intended to mean either a single unitary component or a collection of components that combine to perform a specific function or purpose.

It should be appreciated that in the above description of exemplary embodiments of the invention, various features of the invention are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical, electrical or optical contact, or that two or more elements are not in direct contact with each other but yet still co-operate or interact with each other.

Unless specifically stated otherwise, as apparent from the following discussions, it is appreciated that throughout the specification discussions utilizing terms such as “processing,” “computing,” “calculating,” “determining”, analysing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer” or a “computing machine” or a “computing platform” may include one or more processors.

Some methodologies or portions of methodologies described herein are, in one embodiment, performable by one or more processors that accept computer-readable (also called machine-readable) code containing a set of instructions that when executed by one or more of the processors carry out at least one of the methods described herein. A memory subsystem of a processing system includes a computer-readable carrier medium that carries computer-readable code (e.g., software) including a set of instructions to cause performing, when executed by one or more processors, one of more of the methods described herein. Note that when the method includes several elements, e.g., several steps, no ordering of such elements is implied, unless specifically stated. The software may reside in the storage medium, or may also reside, completely or at least partially, within the RAM and/or within the processor during execution thereof by the computer system. Thus, the memory and the processor also constitute computer-readable carrier medium carrying computer-readable code.

Furthermore, a computer-readable carrier medium may form, or be included in a computer program product.

In alternative embodiments, unless otherwise specified, the one or more processors operate as a standalone device or may be connected, e.g., networked to other processor(s), in a networked deployment, the one or more processors may operate in the capacity of a server or a user machine in server-user network environment, or as a peer machine in a peer-to-peer or distributed network environment. The one or more processors may form a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine.

Note that while only a single processor and a single memory that carries the computer-readable code may be shown herein, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein, unless otherwise specified.

Thus, one embodiment of each of the methods described herein is in the form of a computer-readable carrier medium carrying a set of instructions, e.g., a computer program that is for execution on one or more processors, e.g., one or more processors that are part of web server arrangement. Thus, as will be appreciated by those skilled in the art, embodiments of the present invention may be embodied as a method, an apparatus such as a special purpose apparatus, an apparatus such as a data processing system, or a computer-readable carrier medium, e.g., a computer program product. The computer-readable carrier medium carries computer readable code including a set of instructions that when executed on one or more processors cause the processor or processors to implement a method. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code embodied in the medium.

The software may further be transmitted or received over a network via a network interface device. While the carrier medium may be shown in an embodiment to be a single medium, the term “carrier medium” should be taken to include a single medium or multiple media (for example, a centralized or distributed database, and/or associated caches and servers) that store the one or more sets of instructions. The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media. Non-volatile media includes, for example, optical, magnetic disks, and magneto-optical disks. Volatile media includes dynamic memory, such as main memory. Transmission media includes coaxial cables, copper wire and fibre optics, including the wires that comprise a bus subsystem. Transmission media also may also take the form of acoustic or light waves, such as those generated during radio wave and infrared data communications. For example, the term “carrier medium” shall accordingly be taken to included, but not be limited to, solid-state memories, a computer product embodied in optical and magnetic media; a medium bearing a propagated signal detectable by at least one processor of one or more processors and representing a set of instructions that, when executed, implement a method; and a transmission medium in a network bearing a propagated signal detectable by at least one processor of the one or more processors and representing the set of instructions.

It will be understood that the steps of methods discussed are performed in one embodiment by an appropriate processor (or processors) of a processing (i.e., computer) system executing instructions (computer-readable code) stored in storage.

### INDUSTRIAL APPLICABILITY

The arrangements described are applicable to the medical industry and, and particularly to equipment used in surgical operating theatres and other healthcare environments. Therefore, the invention is clearly industrially applicable.

1. A mobile self-powered medical services trolley for use in an operating theatre, the trolley including:
a portable power supply;
a plurality of instruments integrated onto the trolley and powered by the power supply for use in medical procedures, the plurality of instruments including:
a suction instrument;
a gas supply apparatus; and
wireless data communication unit in communication with a central computer network; and
a multimedia input.

2. The trolley according to claim 1 wherein the power supply includes at least one battery.

3. The trolley according to claim 2 wherein the power supply includes a plurality of batteries connected in parallel.

4. The trolley according to claim 2 wherein the at least one battery is rechargeable.

5. The trolley according to claim 1 wherein the suction instrument includes a vacuum pump.

6. The trolley according to claim 1 wherein the gas supply apparatus includes a carbon dioxide supply apparatus.

7. The trolley according to claim 6 wherein the carbon dioxide supply apparatus includes a carbon dioxide storage cylinder.

8. The trolley according to claim 1 wherein the gas supply apparatus includes at least two gas storage cylinders containing the same medical gas such that the gas supply apparatus is initially connected to one of the at least two gas storage cylinders, the gas supply apparatus configured to automatically change connection to another of the at least two gas storage cylinders when the initially connected gas storage cylinder is depleted.

9. The trolley according to claim 1 wherein the gas supply apparatus includes a plurality of gas storage cylinders, each cylinder containing a different medical gas.

10. The trolley according to claim 1 wherein the multimedia input includes a connector port for connecting to an external video capture device, wherein the connector port is one of: an HDMI port; and an SDI port.

11. (canceled)

12. The trolley according to claim 1 wherein the multimedia input includes an integrated video capture device.

13. The trolley according to claim 11 wherein the integrated video capture device includes at least one video camera.

14. (canceled)

15. The trolley according to claim 1 wherein the wireless data communication unit utilises a wireless ethernet connection.

16. The trolley according to claim 1 wherein the wireless data communication unit includes a transmitter for communicating with a remote receiver connected to the central computer network, the central computer network including a standalone video routing system such that data from the video capture device is inputted into the standalone video routing system for display within the surgical theatre.

17. The trolley according to claim 1 further including a location positioning system for allowing location detection of the trolley.

18. The trolley according to claim 17 wherein the location positioning system includes an integrated radio receiver.

19. The trolley according to claim 1 further including a line isolation safety monitor for monitoring leakage to ground current.

20. A mobile self-powered medical services trolley for use in a surgical theatre, the trolley including:
a portable power supply; and
a plurality of devices integrated onto the trolley and powered by the power supply for use in medical procedures, the plurality of instruments to supply the following functions:
suction;
gas supply;
video capture input; and
wireless data communication.

21. A method for providing self-powered suction, gas supply and video capture to a surgical theatre including providing a medical services trolley according to claim 1.

22. A central computer network for displaying a visual input on at least one monitor within a surgical theatre, the network including a standalone video routing system for receiving video data from a medical services trolley according to claim 1.
